(19) United States
(12) Patent Application Publication
(10) Pub. No.: US 2022/0218608 A1
(43) Pub. Date: Jul. 14, 2022

(54) INJECTABLE POLYMER NANOPARTICLE COMPOSITIONS OF ANTITHROMBOTIC AGENTS AND METHODS THEREOF

(71) Applicant: FORDOZ Pharma Corp., East Windsor, NJ (US)

(72) Inventors: QISHENG XIN, Highland Park, NJ (US); SYDNEY UGWU, North Brunswick, NJ (US); JAMES HE, Green Brook, NJ (US)

(73) Assignee: FORDOZ Pharma Corp., East Windsor, NJ (US)

(21) Appl. No.: 17/606,024

(22) PCT Filed: May 8, 2020
(86) PCT No.: PCT/US2020/032049

§ 371 Date: Oct. 23, 2021

Related U.S. Application Data
(60) Provisional application No. 62/846,058, filed on May 10, 2019.

(51) Int. Cl. A61K 9/107; A61K 9/00; A61K 31/4545; A61K 47/10; A61K 45/06

(52) U.S. Cl. CPC A61K 9/1075; A61K 9/0019; A61K 45/06; A61K 47/10; A61K 31/4545

(57) ABSTRACT

Disclosed are injectable nanoparticle compositions comprising a micelle formulation of an antithrombotic agent and a water-soluble, biodegradable, and amphiphilic polymer that improves water solubility of the antithrombotic agent. A method of preparing the injectable nanoparticle compositions and methods for preventing or treating thrombotic diseases such as venous thromboembolisms and/or stroke using the compositions, as well as devices and kits suitable for such treatment, are also disclosed.

### CROSS-REFERENCE TO RELATED APPLICATION

This application is the U.S. National Stage filing of International Patent Application Number PCT/US2020/032049 filed on May 8, 2020 which claims priority under 35 U.S.C. § 119(e) to U.S. Provisional Application Ser. No. 62/846,058, filed on May 10, 2019, the disclosure of which is incorporated herein by reference in its entirety.

### FIELD OF THE INVENTION

The present disclosure relates to injectable nanoparticle compositions for the prevention or treatment of thrombotic diseases, such as venous thromboembolisms and/or stroke.

### BACKGROUND OF THE INVENTION

Recent drug discovery has led to an increasing number of new drugs with poor water solubility and hence poor bioavailability. The orally administered poorly water-soluble drugs tend to be excreted from the gastrointestinal (GI) tract before being fully dissolved and absorbed into the circulatory system, which usually results in low bioavailability. As a result, increased doses are needed to achieve therapeutic drug concentrations in circulation. Increase in the dose frequently results in unnecessary problems, including GI tract toxicity, issues of inter-patient variability, higher patient costs, inefficient treatment, and increased risks of toxicity, even death.

Poorly water-soluble drugs require safe vehicles for drug solubilization and intravenous infusion. However, large amounts of these vehicles would be required to solubilize the drugs to clinically relevant concentrations, which may lead to toxicity. Examples of such vehicles include surfactants and co-solvents.

Thrombotic diseases, including acute myocardial infarction, unstable angina, deep vein thrombosis, pulmonary embolism, and ischemic stroke, remain the leading cause of morbidity and mortality in the United States and other Western countries. Currently available treatment and prevention therapies include anticoagulants such as vitamin K antagonists (e.g., Warfarin), heparin, Factor Xa inhibitors (e.g., Rivaroxaban, Apixaban), low molecular weight heparins, and antiplatelet agents (e.g., Aspirin and Clopidogrel).

Many of these antithrombotic agents are hydrophobic and have only limited water solubility, providing poor oral bioavailability. There is a need for the development of injectable compositions having improved solubility and a more rapid onset of action for such agents.

### SUMMARY OF THE INVENTION

The present disclosure provides solutions to these identified needs. One aspect of the invention is directed to an injectable nanoparticle composition comprising micelles encapsulating an antithrombotic agent where the antithrombotic agent has a poor water-solubility, wherein the micelle comprises a biodegradable amphiphilic polymer.

The amphiphilic polymer can be selected from, for example, pegylated block copolymers and pegylated phospholipids. The pegylated block copolymer can be selected from, for example, poly(ethylene glycol)-block-polylactide methyl ether (PEG-b-PLA) and poly(ethylene glycol) methyl ether-block-poly(ε-caprolactone) (PEG-b-PCL). The pegylated phospholipid can be selected from, for example, 1,2-distearoyl-sn-glycero-3-phosphoethanolamine-N-[amino(polyethylene glycol)] ammonium or sodium salt (PEG-DSPE).

The antithrombotic agent can be selected from, without limitation, apixaban, rivaroxaban, dabigatran, clopidogrel, prasugrel, prodrugs thereof, and pharmaceutically acceptable salts thereof. In another aspect, the invention is directed to a method of preparing a composition of the invention, comprising the steps of: 1) mixing together an organic solvent, an antithrombotic agent and a biodegradable amphiphilic polymer having a hydrophilic PEG A block component and a hydrophobic B block component, in an amount effective to absorb the antithrombotic agent; 2) evaporating the organic solvent substantially completely to form a gel-like polymer/drug matrix; 3) adding an aqueous medium (e.g., having a pH of 5 to 8) to the gel-like polymer/drug matrix, and mixing gently to form a micelle solution; 4) cooling the micelle solution to room temperature; 5) filtering through a filter (e.g., 0.2 μm) to provide a cooled, filtered micelle solution; 6) adding one or more lyoprotectants to the cooled, filtered micelle solution; and 7) freeze-drying to form a solid state polymeric micellar composition.

Suitable organic solvents include, but are not limited to, methanol, ethanol, isopropanol, butanol, isobutanol, pentanol, dichloromethane, chloroform, acetonitrile, acetone, ethyl acetate, tetrahydrofuran, dimethoxyethane, formic acid, acetic acid, anisole, and a combination of any of two or more thereof.

A further aspect of the invention is directed to a method of treating a thrombotic disease, comprising injecting into a patient in need thereof a therapeutically effective amount of the injectable nanoparticle composition disclosed herein. A related aspect of the invention is directed to a method of preventing a thrombotic disease, comprising injecting into a patient in need thereof a therapeutically effective amount of the injectable nanoparticle composition as disclosed herein. These methods can treat or prevent a thrombotic disease selected from acute myocardial infarction, unstable angina, deep vein thrombosis, pulmonary embolism, or ischemic stroke.

In another aspect of the invention, the present invention provides a solid-state pharmaceutical micelle composition comprising micelles encapsulating an antithrombotic agent, wherein the micelles comprise a biodegradable amphiphilic polymer selected from the group consisting of pegylated block copolymers, pegylated phospholipids, and combinations thereof, in any embodiments as disclosed herein.

In another aspect of the invention, the present invention provides a device or kit containing a pharmaceutical composition disclosed here for convenience of administration to a patient in need of treatment.

Other aspects or advantages of the present invention will be better appreciated in view of the following drawing, detailed description, examples, and claims.

### BRIEF DESCRIPTION OF THE DRAWING

FIG. 1 illustrates the cumulative percent release of Apixaban from different formulations of drug-loaded polymeric micelles into hydroxypropyl-beta cyclodextrin (HP-b-CD) release medium.

### DETAILED DESCRIPTION OF CERTAIN EMBODIMENTS

One aspect of the invention is directed to an injectable nanoparticle composition comprising micelles encapsulating an antithrombotic agent where the antithrombotic agent has poor water-solubility or is essentially insoluble, wherein the micelles preferably comprise a biodegradable amphiphilic polymer.

Biocompatible and biodegradable polymers have been widely used as drug delivery systems. Among them, the amphiphilic polymeric micelles serve as effective vehicles to solubilize and deliver the poorly water-soluble drugs. As defined herein, a polymeric micelle is a nanoparticle having a structure characterized by a hydrophilic shell surrounding a hydrophobic core.

Due to the hydrophobic environment of the core, water insoluble drugs can be easily solubilized to form clear solutions, which are suitable for injection and delivery of the drugs to target tissues. This targeted drug delivery system can minimize drug degradation, reduce drug side effects, increase drug bioavailability and increase the amount of drug delivered to the target site.

The poorly water-soluble drugs suitable for the present invention include a number of known or yet-to-be developed Factor Xa inhibitors.

Factor Xa is a key serine protease in the coagulation cascade and is a promising target enzyme for new therapeutic agents for the treatment and prevention of arterial and venous thrombosis. In particular, factor Xa plays a critical role in blood coagulation, serving as the juncture between the extrinsic (tissue factor initiated) and intrinsic (surface activation and amplification) systems. Factor Xa forms a prothrombinase complex with phospholipids, calcium ions, and a cofactor, factor Va, which complex is responsible for the generation of thrombin from prothrombin. Although factor Xa inhibition attenuates the generation of thrombin, it does not affect thrombin activity, thereby preserving hemostasis, which, in clinical terms, may translate to efficacy with lower bleeding risk.

There has been a great deal of interest in the introduction of novel antithrombotic agents for the prevention and treatment of thrombosis. These novel agents include dabigatran (PRADAXA®) approved by the U.S. Food and Drug Administration (FDA) in 2010, rivaroxaban (XARELTO®) approved in 2011, apixaban (ELIQUIS®) approved in 2012, edoxaban (SAVAYSA®) approved in 2015, and betrixaban (BEVYXXA®, PORTOLA®) approved in 2017. Dabigatran is a direct thrombin inhibitor. Rivaroxaban, apixaban, edoxaban, and betrixaban are all factor Xa inhibitors. These drugs have major pharmacologic advantages over warfarin, which is a vitamin K antagonist, the advantages including rapid onset/offset of action, few drug interactions and predictable pharmacokinetics. While warfarin has a narrow therapeutic window that can be affected by factors such as diet, so an issue for patients taking warfarin is that they need to have their international normalized ratio (INR) monitored regularly.

In January 2019, the atrial fibrillation (AFib) treatment guidelines were updated to indicate that these novel Factor Xa inhibitors are now recommended as the preferred alternative to warfarin for reducing the risk of stroke. This change was made in a focused update to the 2014 American Heart Association (AHA), American College of Cardiology (ACC) and Heart Rhythm Society (HRS) Guideline for the Management of Patients with Atrial Fibrillation.

Most of these newly developed antithrombotic agents are hydrophobic and have only limited water-solubility, which results in low dissolution rate of the API from the pharmaceutical composition and poor oral bioavailability, which can be improved using the present invention. Apixaban, structure shown below, is representative of such hydrophobic and poorly water-soluble antithrombotic APIs.

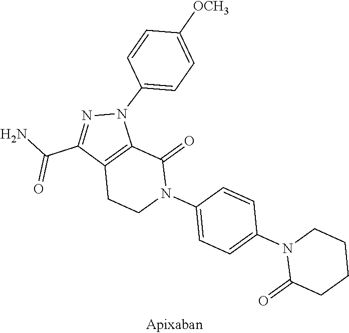 [SMILES: COc1ccc(-n2nc(C(N)=O)c3c2C(=O)N(c2ccc(N4CCCCC4=O)cc2)CC3)cc1]

Furthermore, the time to reach minimum effective concentration in the blood after an oral administration of such drugs is long, 2-4 hours, which delays the desired anticoagulant effects. There is a need for the development of an injectable formulation with an improved solubility and faster onset of action for these agents. This would be particularly useful in clinical emergencies, for example in patients suffering from ischemic stroke.

Other situations in which patients would need an injectable product include, but are not limited to, the following:

1) risk for blood clots when sick or injured and cannot move around very much;

2) blood clot in a blood vessel or lung;

3) certain heart problems or conditions that put the patient at risk for blood clots;

4) certain surgical operations; and

5) patients starting or maintaining warfarin therapy, and the INR blood test results are too low.

In some embodiments, sometimes preferably, the amphiphilic polymer is selected from the group consisting of pegylated block copolymers and pegylated phospholipids.

In some embodiments, sometimes preferably, the pegylated block copolymer is selected from the group consisting of poly(ethylene glycol)-block-polylactide methyl ethers (PEG-b-PLAs), and poly(ethylene glycol) methyl ether-block-poly(ε-caprolactones) (PEG-b-PCLs).

In some embodiments, sometimes preferably, the pegylated phospholipid is selected from 1,2-distearoyl-sn-glycero-3-phosphoethanolamine-N-[amino(polyethylene glycol)] salts (PEG-DSPE) of pharmaceutically acceptable cations, such as ammonium, sodium, or potassium.

In some embodiments, the molecular weight of the poly(ethylene glycol) (PEG) block can be about 1,000 to about 35,000 g/mol, or about 1,500 to about 14,000 g/mol, or about 2,000 to about 12,000, or about 3,000 to about 10,000, or about 4,000 to about 8,000, or about 5,000 to about 7,000 g/mol.

In some embodiments, the molecular weight of the poly(lactic acid) (PLA) or the poly(c-caprolactone) (PCL) block can be about 1,000 to about 15,000 g/mol, or about 1,500 to about 7,000 g/mol, or about 2,000 to about 6,000, or about 2,500 to about 5,000, or about 3,000 to about 4,000 g/mol.

In some embodiments, the biodegradable amphiphilic polymer is present in a range of about 0.1 wt % to about 50 wt % based on the total weight of the composition, or about 0.5 wt % to about 30 wt %, or about 1 wt % to about 25 wt %, or about 2 wt % to about 20 wt % , or about 3 wt % to about 15 wt %, or about 4 wt % to about 10 wt %, or about 5 wt % to about 8 wt % based on the total weight of the composition.

In some embodiments, the antithrombotic agent is selected from the group consisting of apixaban, rivaroxaban, dabigatran, clopidogrel, prasugrel, prodrugs thereof, and pharmaceutically acceptable salts thereof.

In some embodiments, the antithrombotic agent of the injectable nanoparticle composition is present in about 0.1 to about 20 mg/mL of the composition, or about 0.25 to about 10 mg/mL, or about 0.3 to about 4 mg/mL, or about 0.4 to about 3 mg/mL, or about 0.5 to about 2 mg/mL of the composition.

In some embodiments, the amphiphilic polymer : antithrombotic drug ratio in the composition ranges from about 5:1 to about 250:1 (w/w), or about 5:1 to about 200: 1, or about 5:1 to about 150:1, or about 5:1 to about 100:1, or about 5:1 to about 75:1, or about 5:1 to about 50:1; or about 20:1 to about 250:1, or about 25:1 to about 250:1, or about 30:1 to about 250:1, or about 40:1 to about 250:1, or about 50:1 to about 250:1, or about 60:1 to about 250:1, or about 75:1 to about 250:1, or about 100:1 to about 250:1, or about 150:1 to about 250:1, or about 200:1 to about 250:1.

In some embodiments, the organic solvent is selected from the group consisting of methanol, ethanol, isopropanol, butanol, isobutanol, pentanol, dichloromethane, chloroform, acetonitrile, acetone, ethyl acetate, tetrahydrofuran, dimethoxyethane, formic acid, acetic acid, anisole, and a combination of any of two or more thereof.

In some embodiments, the aqueous medium has a pH of about 1 to about 10, about 5 to about 8, sometimes preferably about 5.5 to about 7.5, and some times more preferably about 6 to about 7.5.

In some embodiments, the filter for filtering the micelle solution has an average pore size in the range of about 0.1 μm to about 1.0 μm, sometimes preferably 0.1 μm to about 0.5 μm, sometimes preferably 0.1 μm to about 0.3 μm, sometimes more preferably about 0.15 μm to about 0.25 μm, and sometimes more preferably about 0.2 μm to about 0.22 μm.

In some embodiments, the invention provides a method of preparing a composition of the invention, comprising the steps of: 1) mixing in an organic solvent a pharmaceutically effective amount of an antithrombotic agent with a biodegradable amphiphilic polymer having a hydrophilic PEG A block component and a hydrophobic B block component, in an amount effective to absorb the antithrombotic agent, wherein the organic solvent is selected from the group consisting of methanol, ethanol, isopropanol, butanol, isobutanol, pentanol, dichloromethane, chloroform, acetonitrile, acetone, ethyl acetate, tetrahydrofuran, dimethoxyethane, formic acid, acetic acid, anisole, and a combination of any of two or more thereof; 2) evaporating the organic solvent completely, to form a gel-like polymer/drug matrix where the antithrombotic agent is absorbed in the hydrophobic B block component; 3) adding an aqueous medium having a pH of about 5 to about 8, preferably about 5.5 to about 7.5, more preferably about 6 to about 7.5 to the gel-like polymer/drug matrix, and mixing gently to form a micelle solution; 4) cooling the micelle solution to room temperature; 5) sterilizing by filtering through a 0.2 wn filter to provide a cooled, filtered micelle solution; 6) adding one or more lyoprotectants to the cooled, filtered micelle solution; and 7) freeze-drying the solution to form a solid state polymeric micellar composition.

The aqueous medium of the method preferably comprises water, about 0.5% to about 5.0% (e.g., about 0.7%, 0.9%, 1.0%, 1.5, 2.0%, or 3.0%, or the like) saline, about 5% to about 10% (e.g., 5%, 6%, 7%, 8%, or 9%) sucrose, and about 10 mM to about 100 mM (e.g., 15 mM, 20 mM, 25 mM, 30 mM, 40 mM, 50 mM, 60 mM, 70 mM, 80 mM, or 90 mM) buffer.

In some embodiments, sometimes preferably, the solvent of step 1) comprises a mixture of an alcohol with dichloromethane, and is preferably a mixture of methanol or ethanol with dichloromethane, more preferably a mixture of methanol and dichloromethane.

In one embodiment, the aqueous medium comprises about 0.9% saline, about 5% to about 9% sucrose, and about 15 to about 50 mM buffer.

In some embodiments, the buffering agent is selected from the group consisting of citric acid, acetic acid, ascorbic acid, histidine and salts thereof, sodium citrate, sodium acetate, sodium ascorbate, sodium phosphate monobasic, sodium phosphate dibasic, and combinations thereof.

A further aspect of the invention is directed to a method of treating a thrombotic disease, comprising administering to a patient in need thereof a therapeutically effective amount of the injectable nanoparticle composition in any suitable embodiments disclosed herein.

In one embodiment, the invention provides a method of preventing a thrombotic disease, comprising administering to a subject in need thereof a therapeutically effective amount of the injectable nanoparticle composition as disclosed herein.

In some embodiments, the administering includes, without limitation, injecting to the subject intravenously or parenterally (other than through the intestine), such as subcutaneous (beneath the skin), intramuscular (within the substance of the muscle), and intradermal (within the dermis).

Because of the accuracy of these methods of administration, these injections provide the patient with a more precise amount of drug and a more rapid onset of drug action, as can be more readily determined by a physician.

Thrombotic diseases that may be treated or prevented by using the present invention include, but are not limited to, acute myocardial infarction, angina, deep vein thrombosis (DVT), an embolism, stroke, and the like.

In one embodiment, the thrombotic disease is a pulmonary embolism or ischemic stroke.

In another aspect of the invention, the present invention provides a solid-state pharmaceutical micelle composition comprising micelles encapsulating an antithrombotic agent, wherein the micelles comprise a biodegradable amphiphilic polymer selected from the group consisting of pegylated block copolymers, pegylated phospholipids, and combinations thereof, in any embodiments as disclosed herein.

In another aspect, the present invention is directed to use of a solid-state pharmaceutical micelle composition comprising micelles encapsulating an antithrombotic agent in the manufacture of a medicament for treatment of a thrombotic disease or disorder, wherein the micelles comprise a biodegradable amphiphilic polymer selected from the group consisting of pegylated block copolymers, pegylated phospholipids, and combinations thereof.

The thrombotic disease or disorder include, but are not limited to, acute myocardial infarction, angina, deep vein thrombosis (DVT), an embolism, stroke, and the like. Such use is applicable to all the embodiments of the micelle composition disclosed herein.

In another aspect of the invention, the present invention provides a combination of a device or kit containing a pharmaceutical composition disclosed here for convenience of administration, for example, a syringe containing a single dose of the micellar formulation. Such a syringe can optionally be attached to a needle ready for injection. Such a needle should have a bore size that is appropriate for introduction of the micelles, and may be optionally capped with a needle cover. All such device or kit should be in sterile conditions and preferably stored and readily transportable under such conditions.

As a person of skill in the art would appreciate, all reasonable combinations of the embodiments disclosed, regardless of components or parameters, or otherwise, are encompassed by the present invention.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, various suitable methods and materials are described below. The materials, methods, and examples are illustrative only and not intended to be limiting.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 20” may mean from 18 to 22. Other meanings of “about” may be apparent from the context, such as rounding off, so, for example “about 1” may also mean from 0.5 to 1.4. Similarly, “about 0.2” may encompass a value from 0.18 to 0.22.

As used herein, the singular forms “a,” “an,” and “the” include plural reference, and vice versa, any plural forms include singular reference, unless the context clearly dictates otherwise.

The terms “comprising,” “having,” “including,” and “containing,” or the like, are to be construed as open-ended terms (i.e., meaning “including, but not limited to,”) unless otherwise noted.

As used herein, “micelle” refers to aggregates formed by a biodegradable amphiphilic polymer(s) similarly as surfactants typically form in an aqueous composition, typically when the surfactant is used at a concentration above the critical micelle concentration (CMC). In micelles, the hydrophilic portions of the amphiphilic polymer (surfactant) molecules contact the aqueous or the water phase, while the hydrophobic portions form the core of the micelle, which can encapsulate non-polar (hydrophobic) ingredient(s), for example, a poorly water-soluble drug substance. Typically, the amphiphilic polymer(s) (surfactants) in the provided concentrates form micelles containing the non-polar ingredient at their center in the aqueous liquid dilution compositions.

In one embodiment, the composition of the present invention is self-emulsifying in an aqueous solution. In a further embodiment, the composition forms a micellar dispersion in an aqueous solution.

Suitable biodegradable polymers that may be used for the preparation of micelles of the present invention include, but are not limited to, poly lactic-co-glycolic acid (PLGA), polylactic acid, polycaprolactone (PCL), polyvinyl alcohol, poly(n-isopropylacrylamide), or a combination thereof.

As used herein, the terms “limited water solubility”, “poor water solubility”, “poorly water soluble”, or the like, sometimes used interchangeably, mean that a drug substance (i.e., active pharmaceutical ingredient) has a solubility equal to or less than 1 mg/mL, or 0.5 mg/mL, or 0.2 mg/mL, or 0.1 mg/mL in water at room temperature (about 20 to 22 ° C.).

The term “substantially,” as used herein, means “for the most part” or “essentially”, as would be understood by a person of ordinary skill in the art, for example, in some embodiments, at least 95%, sometimes preferably at least 98.0%, sometimes preferably at least 98.5%, sometimes more preferably at least 99.0%, 99.5%, or 99.8%.

The term “pharmaceutically acceptable” describes a material that is not biologically or otherwise undesirable, i.e., without causing an unacceptable level of undesirable biological effects or interacting in a deleterious manner.

The term “therapeutically effective amount” means an amount effective to deliver a therapeutically effective amount of an amount of active agent needed to delay the onset of, inhibit the progression of, or halt altogether the particular disease, disorder or condition being treated, or to otherwise provide the desired effect on the subject to be treated. As one of ordinary skill in the art would understand, a therapeutically effective amount varies with the patient's age, condition, and gender, as well as the nature and extent of the disease, disorder or condition in the patient, and the dosage may be adjusted by the individual physician (or veterinarian).

The terms “treating” and “treatment” refer to reversing, alleviating, inhibiting, or slowing the progress of the disease, disorder, or condition to which such terms apply, or one or more symptoms of such disease, disorder, or condition.

The term “subject” or “patient” used herein refers to a human patient or a mammalian animal, such as cat, dog, cow, horse, monkey, or the like.

### EXAMPLES

The following examples are illustrative in nature and are not intended to be limiting in any way.

Abbreviations used herein include the following:

mPEG-b-PLA, mPEG-PLA or PEG-PLA: Poly(ethylene glycol)-block-polylactide methyl ether;

mPEG-b-PCL, mPEG-PCL or PEG-PCL: Poly(ethylene glycol) methyl ether-block-poly(ε-caprolactone);

PEG-DSPE: 1,2-distearoyl-sn-glycero-3-phosphoethanolamine-N-methoxy-poly(ethylene glycol) ammonium or sodium salt;

HP-b-CD: Hydroxypropyl-beta cyclodextrin;

INR: International normalized ratio;

Std: standard deviation.
Example 1. General preparation of a polymeric micelle of mPEG-PLA containing Apixaban

A polymeric micellar formulation containing Apixaban was prepared by a method described in Int. J. Pharm., 1996, 132, 195-206, and J. Control. Release, 2001, 72, 191-202, which are hereby incorporated by reference in their entirety.

Briefly, apixaban (7.7 mg) and mPEG-PLA (770 mg, molecular weight =3860-4200 Daltons) were dissolved in 5 mL mixture solvent of methanol/dichloromethane (3/7 v/v). Then, the organic solvent was removed under reduced pressure at 60 ° C. to obtain a transparent gel matrix, which was dissolved by adding 25 mM citrate buffer (pH 6.1) at 60 ° C. to form a transparent apixaban-encapsulated micelle solution. This solution was filtered through a 0.22 μm membrane to sterilize, followed by lyophilization using a freeze dryer system.

The average micelle size was observed to be 19.8 nm, and the Apixaban concentration was 0.5 mg/mL. One or more lyoprotectants were dissolved in the Apixaban solution. The resulting Apixaban-lyoprotectant solutions were then lyophilized under the following conditions: frozen at −40° C. for 4 hours, freeze-dried at −25° C. and 60 mT for 24 hours, and finally freeze-dried at 25° C. for 8 hours. Prior to use, the lyophilized compositions were reconstituted with water.
Example 2. A polymeric micellar composition containing Apixaban was prepared by a method described in Example 1 using the following ingredients:

mPEG-PLA (molecular weight=3860-4200 Daltons), 100 mg.

Apixaban, 1 mg

water, 2 mL.
Example 3. A polymeric micellar composition containing Apixaban was prepared by a method described in Example 1 using the following ingredients:

mPEG-PLA (molecular weight=3860-4200 Daltons), 100 mg

Apixaban, 1 mg

25 mM citrate buffer (pH 6.1), 2 mL.

Sucrose: 222 mg.
Example 4. A polymeric micellar composition containing Apixaban was prepared by a method described in Example 1 using the following ingredients:

mPEG-PLA (molecular weight=3860-4200 Daltons), 100 mg.

Apixaban, 1 mg

25 mM citrate buffer (pH 6.1), 2 mL.

Sucrose: 105 mg.
Example 5. A polymeric micellar composition containing Apixaban was prepared by a method described in Example 1 using the following ingredients:

mPEG-PLA (molecular weight=3860-4200 Daltons), 100 mg.

Apixaban, 1 mg

25 mM citrate buffer (pH 6.1), 2 mL.

mPEG2000: 105 mg.
Example 6. A polymeric micellar composition containing Apixaban was prepared by a method described in Example 1 using the following ingredients:

mPEG-PLA (molecular weight=3860-4200 Daltons), 100 mg.

Apixaban, 1 mg

25 mM citrate buffer (pH 6.1), 2 mL.

PEG400: 105 mg.
Example 7. A polymeric micellar composition containing Apixaban was prepared by a method described in Example 1 using the following ingredients:

mPEG-PLA (molecular weight=3860-4200 Daltons), 100 mg.

Apixaban, 1 mg

25 mM citrate buffer (pH 6.1), 2 mL

HP-b-CD: 105 mg.
Example 8. Physical Characterization

Lyophilized polymeric micellar compositions were reconstituted in water. Particle size and osmolality were determined by dynamic light scattering and freezing point osmometric methods, respectively. As shown in Table 1, the mean particle size of the various compositions ranged from 20 to 28 nm. The mean size remained unchanged after lyophilization. The osmolality values ranged from 193 to 512 mOsm/kg.

TABLE 1
Summary of Physicochemical Parameters of Apixaban Composition Examples

| | Amount of | Amount of | | | | |
|---|---|---|---|---|---|---|
| | Apixaban | mPEG-PLA | Lyoprotectant | Osmolality | Mean Size (std) (nm) | |
| Example | added (mg) | added (mg) | added (mg) | (mOsm/kg) | before lyo | after lyo |
| 2 | 1 | 100 | — | — | 16.0 (2.9) | — |
| 3 | 1 | 100 | 222, sucrose | 512 | 24.2 (9.8) | 24.4 (7.0) |
| 4 | 1 | 100 | 105, sucrose | 316 | 20.7 (7.6) | 20.9 (5.6) |
| 5 | 1 | 100 | 105, mPEG 2000 | 234 | 28.2 (13.1) | 27.8 (8.8) |
| 6 | 1 | 100 | 105, PEG 400 | 359 | 22.0 (9.3) | 23.3 (10.0) |
| 7 | 1 | 100 | 105, HP-b-CD | 193 | 28.2 (18.8) | 25.5 (9.0) |

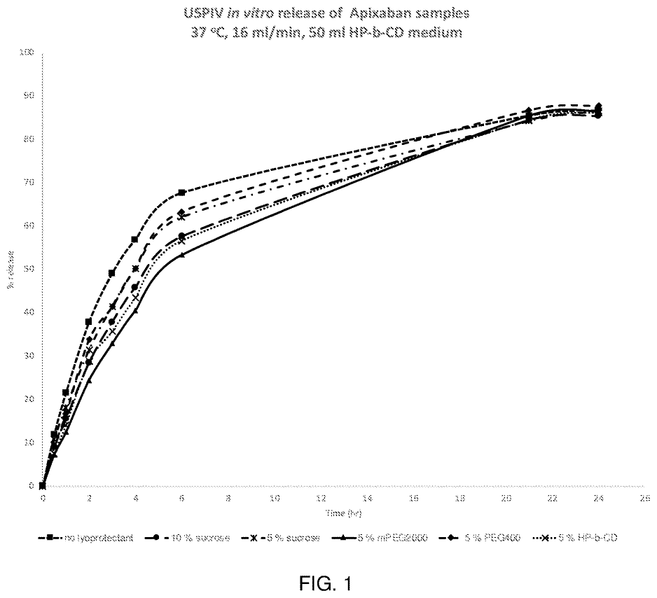

FIG. 1 shows the in vitro release profiles for the inventive polymeric micelle preparations containing mPEG-PLA polymer and lyoprotectants, represented by Examples 2 through 7 (see Table 1). The release medium is 10% HP-b-CD solution. FIG. 1 demonstrates that 50% to 60% of Apixaban was released in an approximately linear fashion for up to 6 hours. In addition, greater than 80% Apixaban was released within 24 hours.
Example 9. Stability Analysis and Results

Lyophilized polymeric micellar compositions were stored at 2-8° C. for 1, 2 3, 6 and 12 months. The lyophilized polymeric micellar compositions were reconstituted in water. Particle size and osmolality were determined by dynamic light scattering and freezing point osmometric methods, respectively. Apixaban concentration was determined by HPLC method. Results are summarized in Table 2.

TABLE 2
Summary of Physicochemical Parameters of Apixaban Composition
after storage at 2-8 C. for 1, 2, 3, 6 and 12 months.

| | | Physical | Appearance | | | | | Apixaban |
|---|---|---|---|---|---|---|---|---|
| | | appearance | after | Size nm | | Osmolality | | (as % of |
| Examples | Months | of lyo cake | reconstitution | (std) | | (mOsm/kg) | pH | initial conc) |
| Example 1 | 1 | white cake | clear | 18.7 | (5.6) | 144 | 5.81 | 99.8 |
| | 2 | white cake | clear | 17.4 | (4.6) | 154 | 5.98 | 99.1 |
| | 3 | white cake | clear | 17.7 | (6.4) | 150 | 5.87 | 99.4 |
| | 6 | white cake | clear | 19.2 | (6.9) | 154 | 5.91 | 99.2 |
| | 12 | white cake | clear | 18.8 | (5.9) | 132 | 5.9 | 99.0 |
| Example 4 | 1 | white cake | clear | 21.3 | (7.2) | 301 | 5.72 | 99.7 |
| | 2 | white cake | clear | 21.4 | (7.3) | 321 | 5.92 | 99.5 |
| | 3 | white cake | clear | 19.9 | (6.1) | 322 | 5.92 | 99.5 |
| | 6 | white cake | clear | 21.5 | (6.8) | 319 | 5.95 | 99.4 |
| | 12 | white cake | clear | 21.8 | (7.2) | 292 | 5.9 | 99.4 |
| Example 6 | 1 | white cake | clear | 23.7 | (9.8) | 330 | 5.85 | 99.5 |
| | 2 | white cake | clear | 23.3 | (9.9) | 347 | 5.88 | 99.0 |
| | 3 | white cake | clear | 24.4 | (12.5) | 351 | 6.01 | 99.3 |
| | 6 | white cake | clear | 21.5 | (11.8) | 354 | 5.96 | 99.2 |
| | 12 | white cake | clear | 25.4 | (13.4) | 309 | 6.05 | 99.3 |

It will be understood by those of skill in the art that numerous and various modifications can be made without departing from the spirit of the present invention. Therefore, it should be understood that the various embodiments of the present invention described herein are illustrative only and are not intended to limit the scope of the present invention. All literature references cited are incorporated by reference in their entireties.

1-21. (canceled)

22. An injectable nanoparticle composition comprising micelles encapsulating an antithrombotic agent, wherein the micelles comprise a biodegradable amphiphilic polymer.

23. The composition of claim 22, wherein the biodegradable amphiphilic polymer is selected from the group consisting of pegylated block copolymers, pegylated phospholipids, and combinations thereof.

24. The composition of claim 23, wherein the pegylated block copolymer is selected from the group consisting of polyethylene glycol)-block-polylactide methyl ether (PEG-b-PLA) and poly(ethylene glycol) methyl ether-block-poly(ε-caprolactone) (PEG-b-PCL).

25. The composition of claim 23, wherein said pegylated phospholipid is selected from 1,2-distearoyl-sn-glycero-3-phosphoethanolamine-N-[amino(polyethylene glycol)] ammonium or sodium salt (PEG-DSPE).

26. The composition of claim 24, wherein the molecular weight of the poly(ethylene glycol) (PEG) block is about 1,000 to about 35,000 g/mol, and the molecular weight of the poly(lactic acid) (PLA) or the poly(ε-caprolactone) (PCL) block is about 1,000 to about 15,000 g/mol.

27. The composition of claim 24, wherein the molecular weight of the polyethylene glycol) (PEG) block is about 1,500 to about 14,000 and the molecular weight of the polylactic acid) (PLA) or the poly(c-caprolactone) (PCL) block is about 1,500 to about 7,000 g/mol.

28. The composition of claim 22, wherein the biodegradable amphiphilic polymer is present in a range of about 0.1 wt % to about 50 wt % based on the total weight of the composition.

29. The composition of claim 22, wherein the biodegradable amphiphilic polymer is present in a range of about 0.5 wt % to about 30 wt % based on the total weight of the composition.

30. The composition of claim 22, wherein the antithrombotic agent is selected from the group consisting of apixaban, rivaroxaban, dabigatran, clopidogrel, and prasugrel, or a pharmaceutically acceptable salt or prodrug thereof.

31. The composition of claim 22, wherein the concentration of the antithrombotic agent is in the range of about 0.1 mg/mL to about 20 mg/mL of the composition.

32. The composition of claim 22, wherein the antithrombotic agent is in the range of about 0.25 mg/mL to about 10 mg/mL of the composition.

33. The composition of claim 22, wherein the amphiphilic polymer: antithrombotic drug ratio in the composition ranges from about 5:1 (w/w) to about 250:1 (w/w).

34. A method of preparing an injectable nanoparticle pharmaceutical composition of claim 22, comprising the step of mixing in an organic solvent an antithrombotic agent with a biodegradable amphiphilic polymer comprising a hydrophilic PEG A block component and a hydrophobic B block component in amounts sufficient to absorb the antithrombotic agent to form micelles encapsulating the antithrombotic agent.

35. The method of claim 34, wherein the organic solvent is selected from the group consisting of methanol, ethanol, isopropanol, butanol, isobutanol, pentanol, dichloromethane, chloroform, acetonitrile, acetone, ethyl acetate, tetrahydrofuran, dimethoxyethane, formic acid, acetic acid, anisole, and combinations thereof.

36. The method of claim 34, further comprising the steps of:
1) evaporating the organic solvent to form a gel-like polymer/drug matrix;
2) adding an aqueous medium having a pH in the range of 1-10 to the gel-like polymer/drug matrix, and mixing to form a micelle suspension;
3) cooling the micelle suspension to a temperature of about 2-25° C.;
4) filtering through a filter to provide a cooled, filtered micelle suspension;
6) adding one or more lyoprotectants to the cooled, filtered micelle suspension; and
7) freeze-drying to form a solid state polymeric micellar composition.

37. The method of claim 36, wherein the aqueous medium comprises water, about 0.5-5% saline, about 1% to 10% sucrose, and about 10 to 100 mM citrate buffer.

38. A method of treating a thrombotic disease, comprising administering to a subject in need thereof a therapeutically effective amount of an injectable nanoparticle composition of claim 22.

39. The method of claim 38, further comprising the administration of a second therapeutic agent for thrombotic disease.

40. The method of claim 39, wherein the second therapeutic agent is a thrombolytic agent selected from the group consisting of eminase (anistreplase), streptase (streptokinase, kabikinase), reteplase (r-PA or retavase), alteplase (t-PA or activase), TNKase (tenecteplase), abbokinase, kinlytic (rokinase), urokinase (Abbokinase), prourokinase, and anisoylated purified streptokinase activator complex (APSAC).

41. The method of claim 38, wherein the thrombotic disease is acute myocardial infarction, unstable angina, deep vein thrombosis, pulmonary embolism, or ischemic stroke.
